(19) United States
(12) Patent Application Publication
(10) Pub. No.: US 2010/0145191 A1
(43) Pub. Date: Jun. 10, 2010

(54) Method of Marking Biological Tissues for Enhanced Destruction by Applied Radiant Energy

(75) Inventor: Steven D. Jensen, South Jordan, UT (US)

Correspondence Address: CAO Group, Inc., 4628 W. Skyhawk Drive, West Jordan, UT, 84084, US

(73) Assignee: CAO GROUP, INC., West Jordan, UT (US)

(21) Appl. No.: 12/563,211

(22) Filed: Sep. 21, 2009

Related U.S. Application Data
(63) Continuation-in-part of application No. 11/423,424, filed on Jun. 9, 2006, now abandoned.
Application No. 11/423,424 is a continuation of application No. 12/555,692, filed on Sep. 8, 2009.

(51) Int. Cl. A61B 5/05

(52) U.S. Cl. 600/426

(57) ABSTRACT

Methods for staining a selected tissue with a dye, stain or pigment that is attuned to absorb the energy from a radiant energy source are disclosed. The stain enhances absorption of incoming radiant energy, which results in increased destruction of stained tissues and decreased destruction of underlying tissues. This method provides clinicians with the ability to selectively mark a tissue for destruction, while leaving wanted tissues generally intact. Optionally, a radiant energy opaque substance that can be applied adjacent the stained treatment area to protect against incidental exposure to untargeted tissue. Also optionally, an oxidizing substance may be applied with the stain to further enhance the effect of this method. Wavelengths of radiant energy to which tissue is normally transparent may be utilized by applying appropriate stains to targeted tissue, thereby allowing targeted tissue to even be destroyed when it lies beneath untargeted tissue.

### CROSS-REFERENCES TO RELATED APPLICATIONS

The present Application claims priority as a continuation-in-part application of prior U.S. application Ser. No. 11/423,424, filed Jun. 6, 2006 and U.S. application Ser. No. 12/555,692, filed Sep. 8, 2009. Both Applications are incorporated herein in their entirety by reference.

### FIELD OF THE INVENTION

The present invention relates to the field of laser treatment of biological tissues and more particularly relates to a method of staining or dying selected tissues for destruction by a general laser source rather than selecting a particular laser source dependent upon the type of tissue to be treated.

### BACKGROUND OF THE INVENTION

Clinicians are often confronted with the need of a device to cut or destroy various tissues. In recent years, lasers have become a more common device in the hands of medical, dental and veterinary practitioners. Lasers are effective tools for surgical removal of unwanted tissues or routine incisions. One of many advantages of laser surgery is the cauterizing effect of laser treated tissues, which creates a bloodless surgical environment.

There are various types of lasers with multiple wavelengths and power outputs currently manufactured to best fit particular surgical needs. A practitioner will most likely choose a laser, or a number of lasers, that will cover as many routine procedures as possible. There are many reasons that any laser is selected for purchase, namely applicability to the physician's practice, cost, ease of use, size of unit, wavelength and power output. Most lasers produce bands of coherent light of a very narrow wavelength. This narrow wavelength is a major limitation of the current generation of lasers. It requires that a specific type of laser must be chosen that emits a wavelength that is absorbed by a particular biological substrate; therefore, there are many types of lasers manufactured that individually cover a small portion of the electromagnetic spectrum. Each type of laser will then have a different clinical use or application than another type. This results in the clinician having more than one laser in order to adequately perform various biological procedures. There is particular effectiveness of this method in dental procedures, such as a caries treatment, gingivalectomy or root canal treatment, and surface dermatological treatments, such as a mole removal.

The use of a laser, however, does present one disadvantage. Since the laser is attuned to a narrow wavelength range, it is rare that the range will correspond to the most efficiently absorbed wavelength of subjected tissues.

Two main situations cause this disadvantage. The first situation is that different layers of biological tissues that may need incised or treated in the same procedure will be attuned to different wavelengths, thus necessitating a laser that will treat all layers somewhat efficiently, but never precisely. This necessity results in excess energy being used to treat, or just get through, less efficiently absorbing tissue while more efficiently absorbing underlying tissue is bombarded with energy the exterior layer did not absorb. Secondly, different people will have different shades of tissue, in particular skin tone, when compared to others and on various parts of their own bodies (i.e. moles). One laser is not going to be attuned to all of these variations and, even if one were attuned to one particular patient's tissue, its effectiveness would change on the next patient and, possibly, at the instant a procedure was complete (e.g. a mole removal) before the laser could be shut down. In either case, the imprecise attunement of the laser to the tissue causes some degree of overpenetration. Overpenetration is the exposure, and destruction, of a column of tissue underlying the targeted tissue to unabsorbed radiant energy as it spills into deeper biological layers. Overpenetration typically causes a blistering effect as fluid released from the unwanted destruction of tissues is expressed through the wound caused by the procedure.

The present invention is a method of staining a given biological substrate for attunement to a given laser source, rather than the other way around as is practiced in the prior art. When employed with the methods disclosed herein, any efficient laser can be used on any biological substrate regardless of the wavelengths produced. The use of a stain also concentrates the laser's radiant energy in the stained tissues, lessening overpenetration by forcing an attunement of the tissues to the laser output. In addition, a substance that is opaque to a particular radiant energy can be applied around the stained treatment area to protect against incidental or accidental exposure of laterally located tissues to harmful radiant energy during treatment. Given the cost advantage of producing and purchasing a stain over a laser, the method of the present invention represents an extremely cost beneficial advancement in the art.

### SUMMARY OF THE INVENTION

The present invention is a method for destroying, specifically vaporizing or carbonizing, tissue by first applying a dye, stain or pigment to biological tissues that is attuned to absorb incoming radiant energy, which results in the destruction of said tissues. As examples, the dye, stain or pigment could be indocyanine green, carbon black, FD&C Blue #2, nigrosin or others. The dye, stain or pigment may be applied by a pen, a brush, spraying, a fibrous pellet, a syringe tip, fiber syringe tip, or otherwise. The radiant energy source can be any source whose energy is absorbed by the dye, pigment or stain in order to build up heat, such as a diode laser, a gas laser, a solid state laser, non-coherent light, incandescent light, light emitting diode, plasma arc light, halogen bulb, electron beam, or otherwise. It is preferred to use a laser light source due to the inherent efficiency of laser light in this procedure. If desired, an opaque substance may be used to protect tissues, which are not to be cut or destroyed. Opaque substances could include titanium dioxide, zinc oxide, calcium carbonate, or otherwise.

The present invention represents a departure from the prior art in that the method of the present invention dictates the staining of a selected tissue with a dye, stain or pigment. The stain is selected because it is attuned to absorb the energy from a given radiant energy source, rather than selecting a laser source for a particular biological substrate as is current practice. The radiant energy source is then sufficient to destroy or carbonize stained tissues, which are attuned to absorb the energy from the source by the stain. The stain enhances absorption of incoming radiant energy, which results in increased and accelerated destruction of stained tissues.

The increased absorption by stained tissues then reduces overpenetration into the column of tissues underlying the stained tissue. Therefore, this method provides clinicians with the ability to selectively mark a tissue for destruction, while leaving wanted tissues generally intact. The method also allows the most efficient laser to be used on any biological substrate regardless of the wavelengths produced. For example, a stain may be applied in a liquid form directly to selected biological tissues, followed by radiating the stained area with a laser that produces a wavelength that the stain readily absorbs. The method also incorporates the use of a radiant energy opaque substance that can be applied adjacent the stained treatment area to protect against accidental or incidental exposure to wanted tissue.

The more important features of the invention have thus been outlined in order that the more detailed description that follows may be better understood and in order that the present contribution to the art may better be appreciated. Additional features of the invention will be described hereinafter and will form the subject matter of the claims that follow.

Many objects of this invention will appear from the following description and appended claims, reference being made to the accompanying drawings forming a part of this specification wherein like reference characters designate corresponding parts in the several views.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIGS. 1 through 17 are graphs of absorption spectra, depicting absorption intensity over light wavelength of sample stains, each figure and stain being listed below.

FIG. 1 is an absorption spectrum graph of amaranth.

FIG. 2 is an absorption spectrum graph of 8-anilinonaphthalene-1-sulfonic acid ammonium salt.

FIG. 3 is an absorption spectrum graph of bromophenol red (ph7).

FIG. 4 is an absorption spectrum graph of cresol red.

FIG. 5 is an absorption spectrum graph of 2, 7 dichlorofluroescein.

FIG. 6 is an absorption spectrum graph of eosin 4-isothiocyanate.

FIG. 7 is an absorption spectrum graph of eosin Y.

FIG. 8 is an absorption spectrum graph of FD&C Blue #1.

FIG. 9 is an absorption spectrum graph of FD&C Green #3.

FIG. 10 is an absorption spectrum graph of FD&C Yellow #5 (Tartrazine).

FIG. 11 is an absorption spectrum graph of methylene blue.

FIG. 12 is an absorption spectrum graph of naphthol blue black.

FIG. 13 is an absorption spectrum graph of nigrosin.

FIG. 14 is an absorption spectrum graph of neutral red.

FIG. 15 is an absorption spectrum graph of safranine O.

FIG. 16 is an absorption spectrum graph of thymol blue.

FIG. 17 is an absorption spectrum graph of xylenol blue.

FIG. 18 is a sectional view depicting an alternate embodiment of the invention, targeting sub-dermal tissue.

### DEFINITIONS USED IN THE SPECIFICATION

As used in this Application, the term “carbonize” shall mean “to apply energy to organic matter until it turns into carbon and/or oxides resulting from combustion.” The term “vaporize” shall mean “to convert an object or compound into vapor.” All three processes will occur in a tumor or tissue subjected to the methods described in this Application and this Application specifically and exclusively deals with the destruction of undesired tissue through these processes. Accordingly, as used in this Application, the term “destroy”, then, shall mean “destroy through carbonization and/or vaporization. This definition shall be to the exclusion of any other methods of destruction.

This Application shall use the term “stain” to include all such dyes, pigments and stains and any compound or solution utilizing such dye, pigment or stain as an ingredient in its combined whole. The use of the term “stain” is to be understood to include such “stains” that include a pigment or dye as its only ingredient.

The Application specifically deals with the destruction of unwanted or diseased tissue. “Tissue” shall be defined as an aggregate of similar cells and cell products forming a definite kind of structural material with a specific function, in a multi-cellular organism.

These definitions are used throughout this entire Specification and the appended claims.

### DETAILED DESCRIPTION OF THE PREFERRED EMBODIMENT

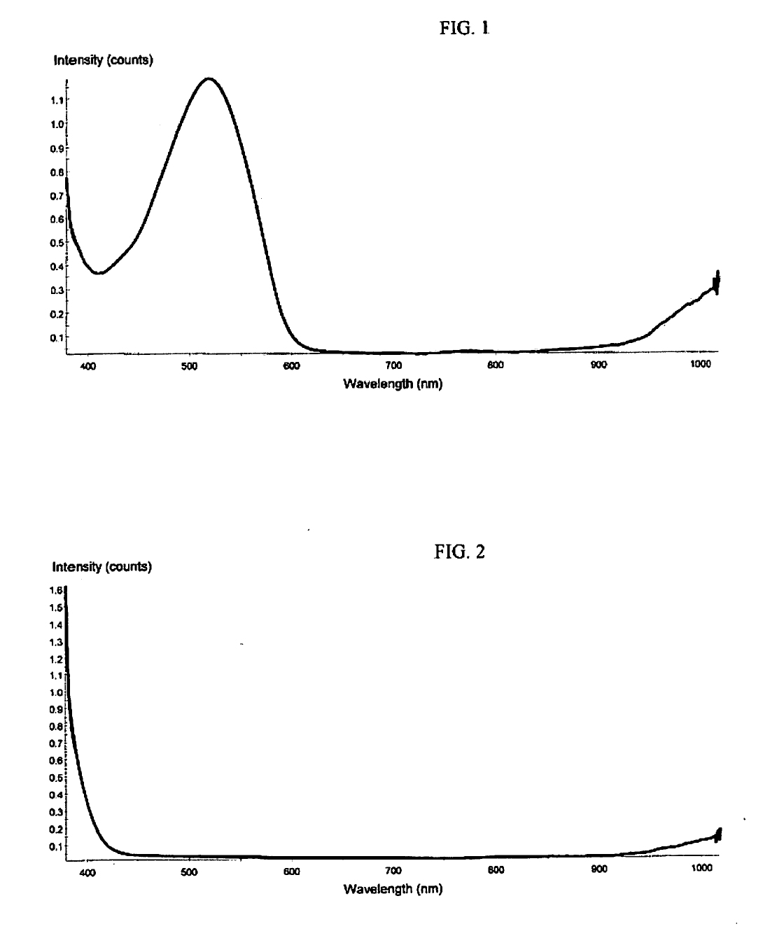

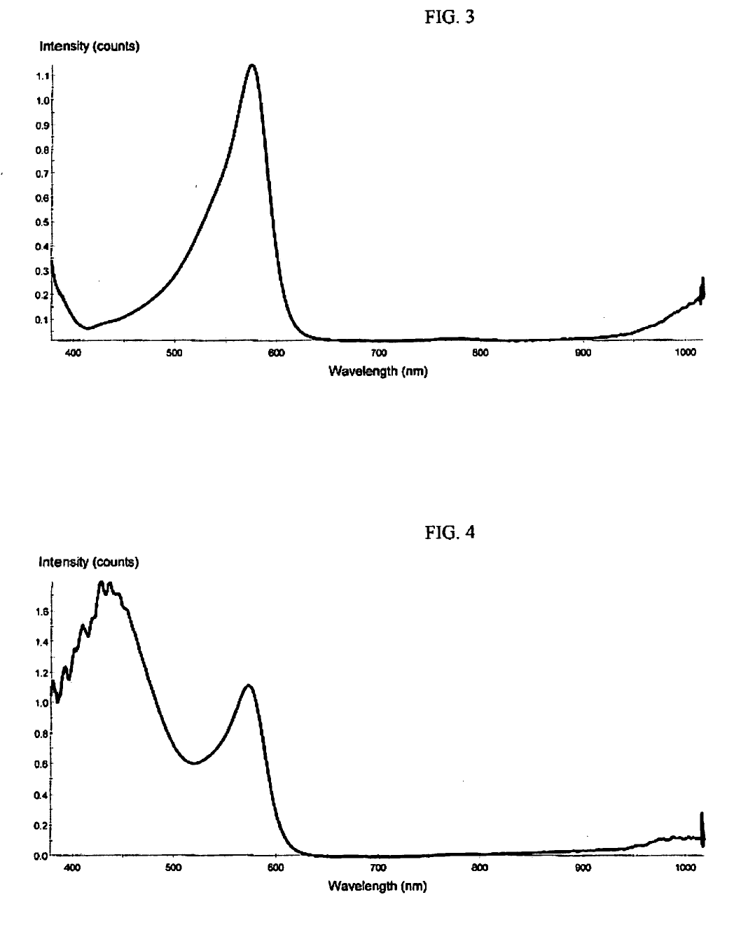

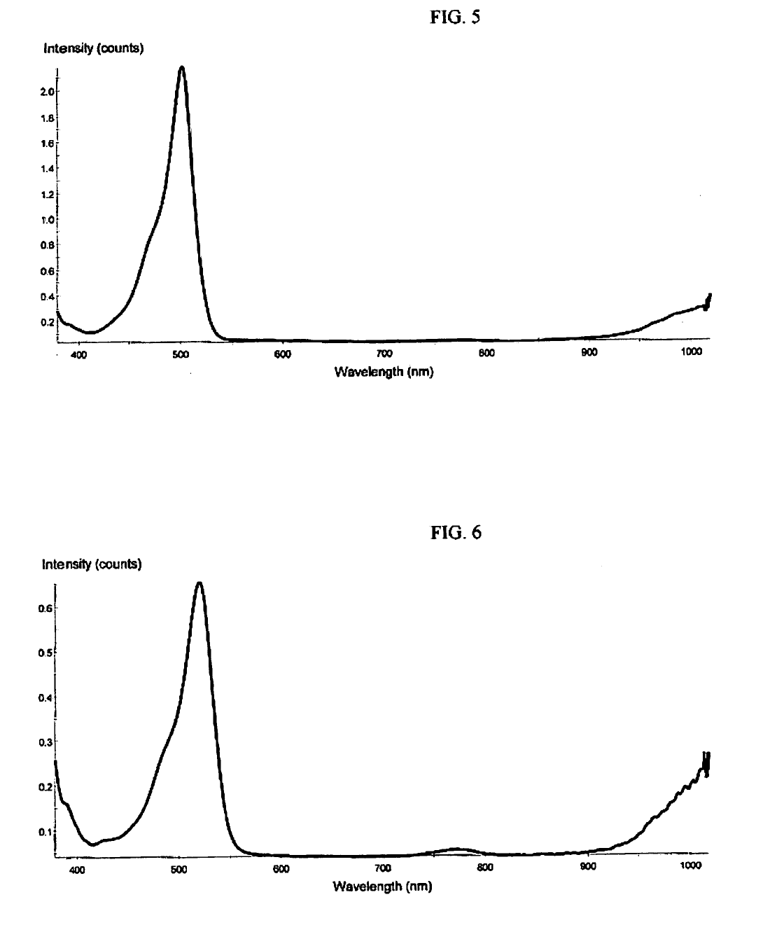

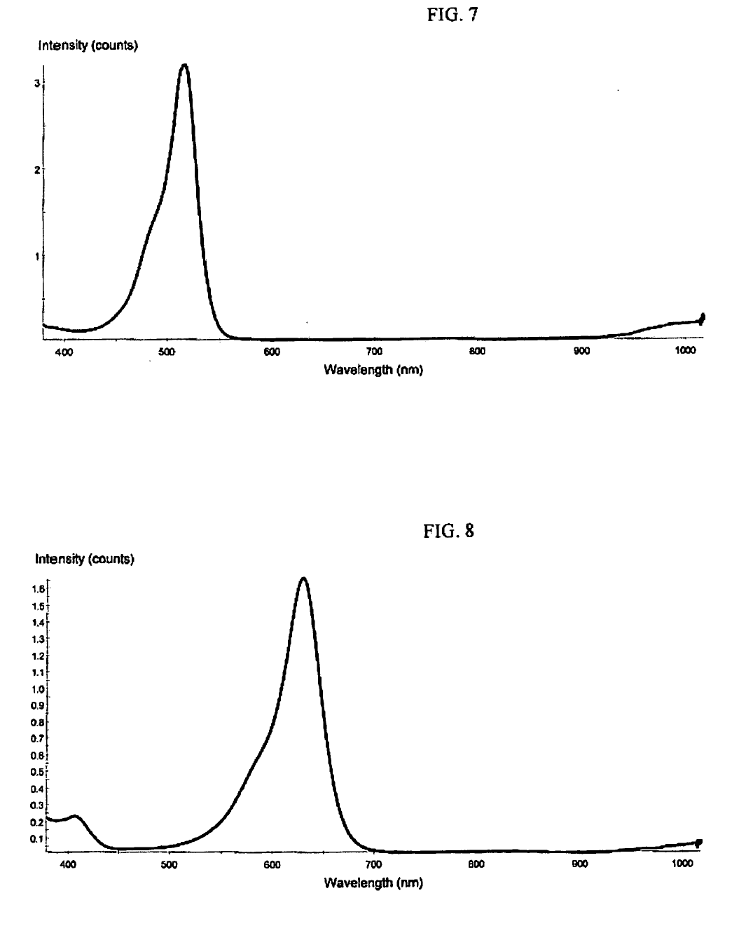

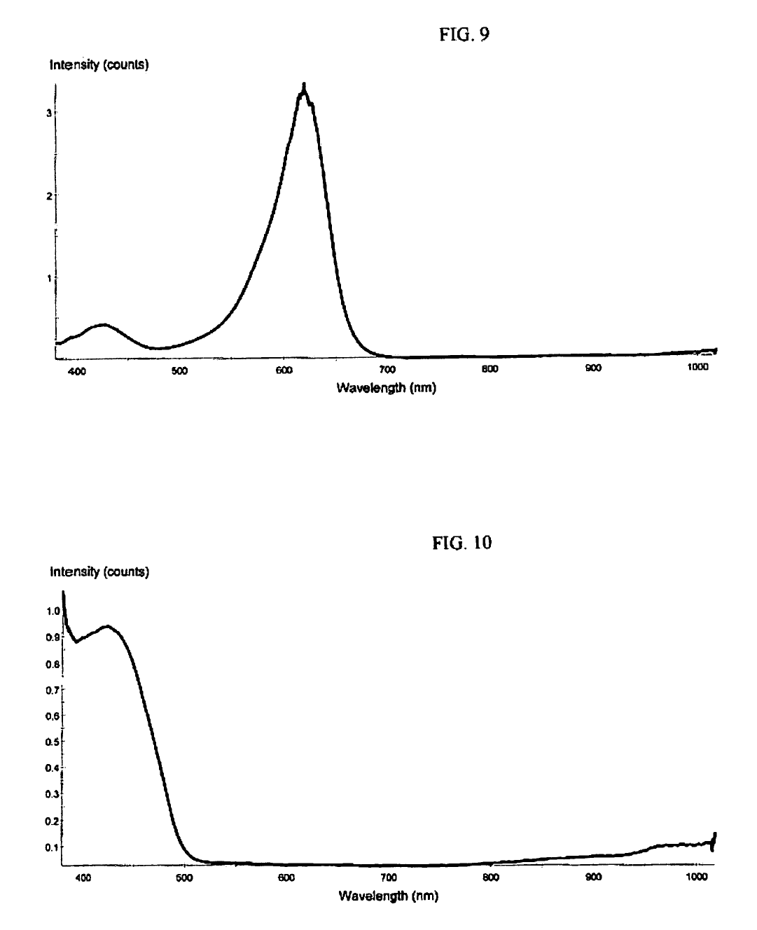

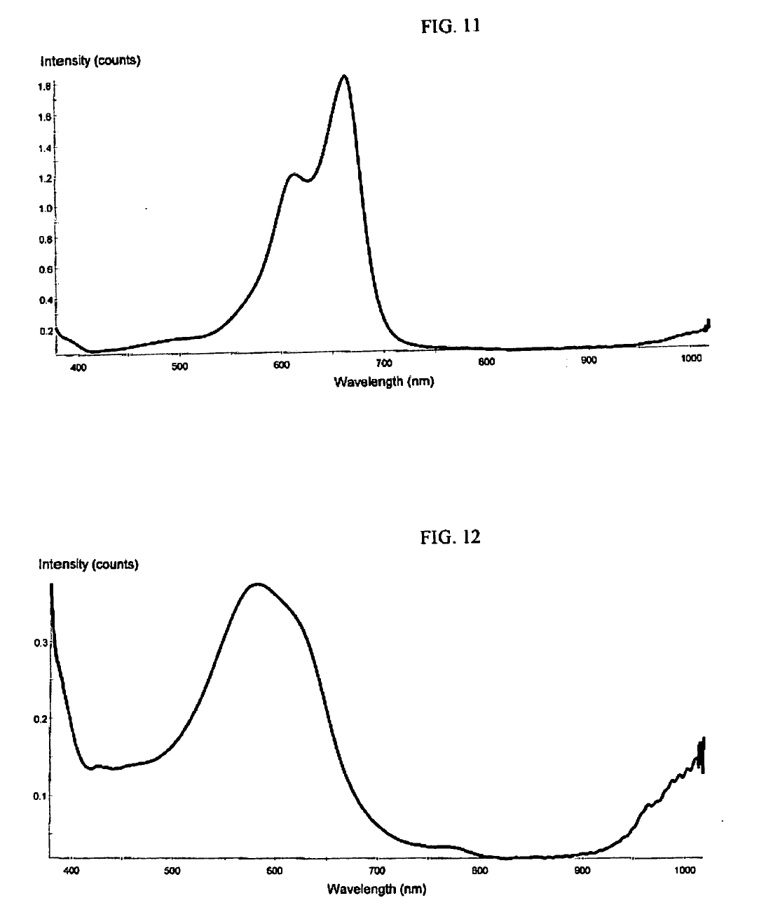

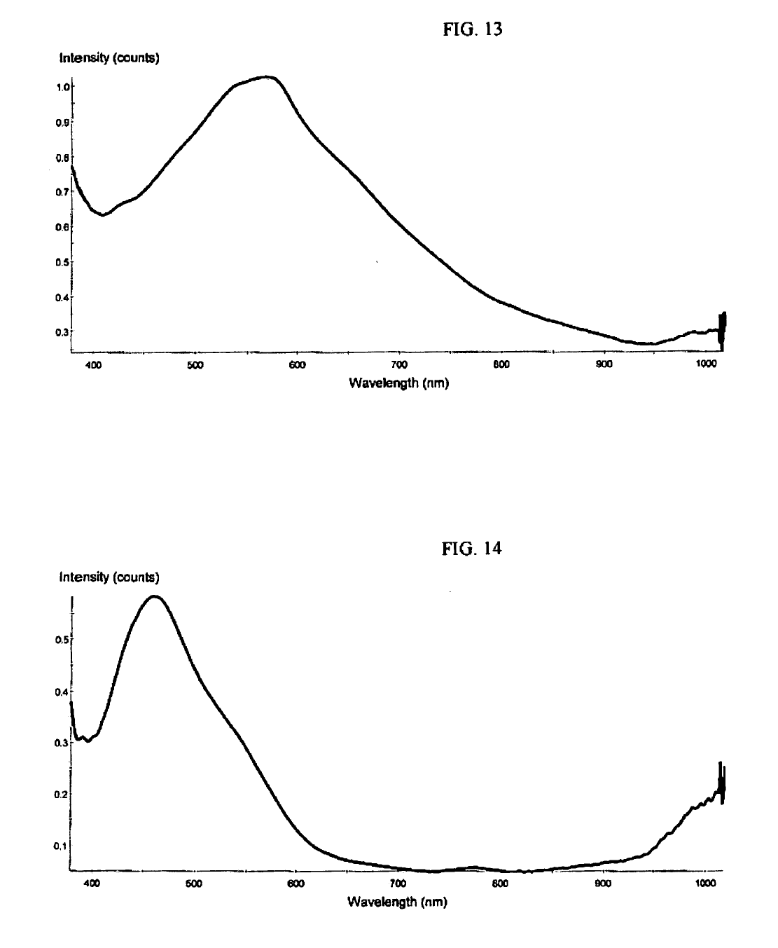

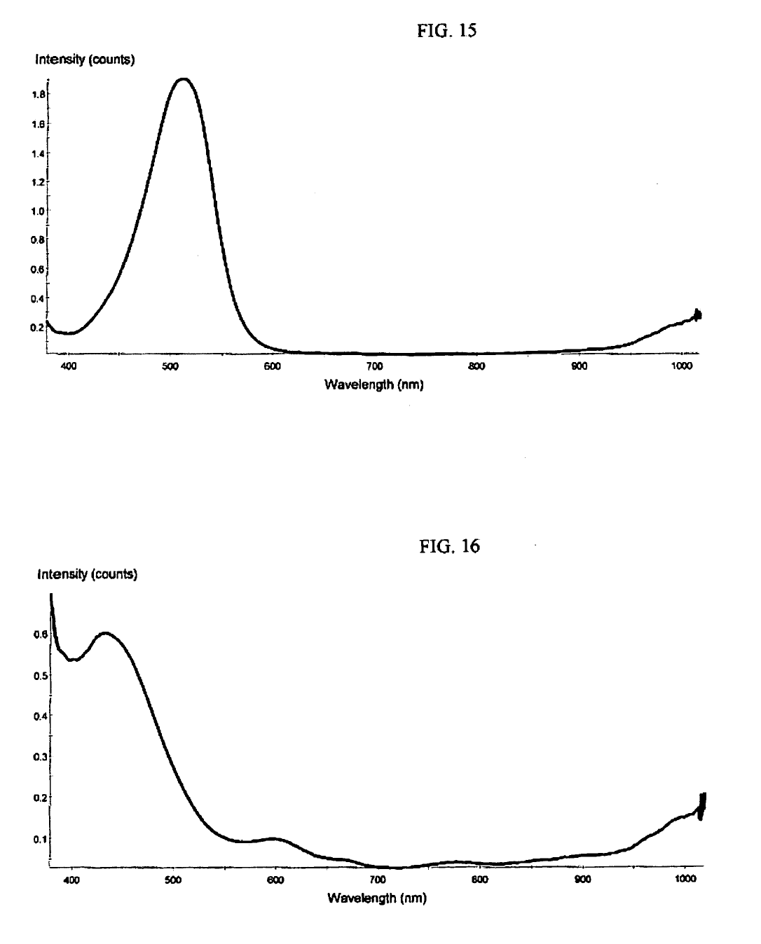

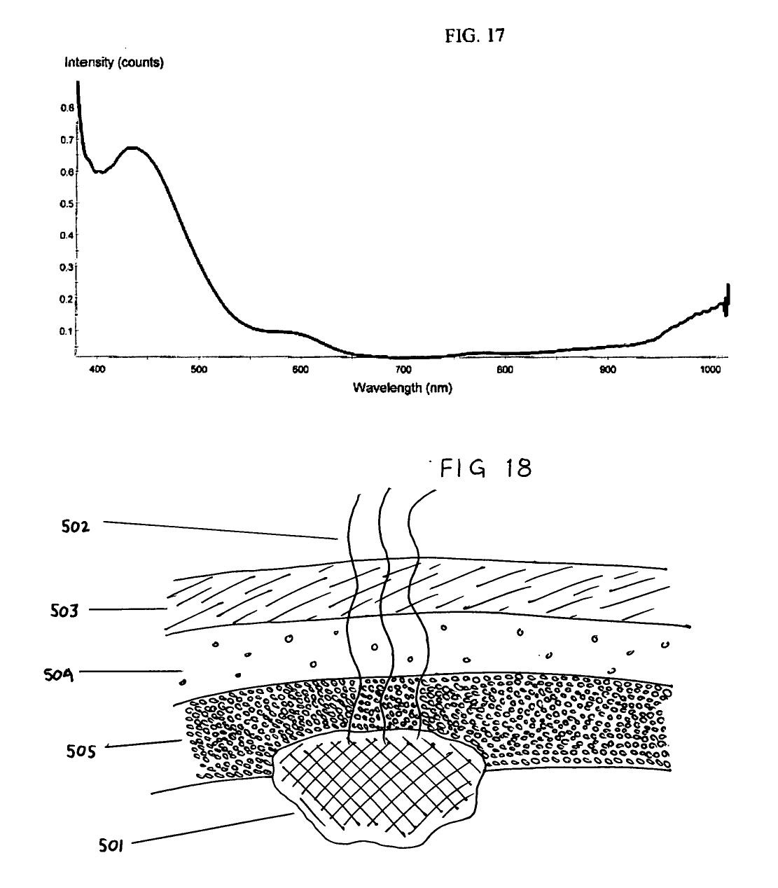

With reference now to the drawings, the preferred embodiment of the method is herein described. It should be noted that the articles “a”, “an” and “the”, as used in this specification, include plural referents unless the content clearly dictates otherwise. FIGS. 1-17 are examples of absorption spectra of various stains that could be used in the disclosed method. Comparing absorption spectra with the wavelength of a radiant energy source permits matching the source and stain for an efficient tissue cutting system and method. As shown in FIG. 1, the absorption spectrum for amaranth peaks at a wavelength of approximately 510 nm, the λₘₐₓ. Therefore, the use of a radiant energy source that has an energy output of 510 nm with the dye amaranth would be in accordance with the method herein disclosed. Likewise, FIGS. 2 through 17 are the spectra for sixteen other stains, each having at least one λₘₐₓ and each may be utilized with an energy source with an output having a wavelength corresponding to a given stain's λₘₐₓ.

In a particular example of the practice of this method, it should be noted that diode lasers are capable of emitting energy with a wavelength of 810 nm. Indocyanine green, a particular stain that has been used extensively in other, unrelated, medical applications, has a λₘₐₓ of approximately 810 nm. The use of indocyanine green as an enhancing stain to aid in procedures where the practitioner uses a diode laser is firmly within the teachings of this method.

The method includes staining a selected tissue with a stain that is attuned to absorb the energy from a radiant energy source. The stain enhances absorption of incoming radiant energy, which results in increased destruction of stained tissues and the lessening of destruction of the column of tissues underneath the stained tissue. This method allows biological tissues to be destroyed by various strategies. Radiant energy can be concentrated to a degree as to totally annihilate a targeted biological tissue. Radiant energy can also be applied to damage tissues sufficiently that it will ultimately result in a scab, and be removed by natural events. The stain can be comprised of any substance with the ability to absorb or accept electromagnetic radiation from any radiant energy source. Radiant energy may be applied to an area from inside, arthroscopically, or outside the body.

In relation to tumors, which acquire their own blood supply independent from host tissue, injection of a stain into the independent supply stains the tumor alone and leaves the surrounding tissue unaltered. Treatment by radiant energy located anywhere in relation to the body may then be utilized. It should also be noted that some biological tissues are transparent to some forms of radiant energy, e.g. flesh vis-a-vis X-rays. In theory, it is possible to stain a biological tissue, even a tumor, so that it is no longer transparent to a particular form of energy and use that energy to treat the stained tissue without harming the surrounding, or even overlying, tissue.

There are literally thousands of dyes, stains and pigments that are commercially available and could be used with the disclosed methods. A few examples of such dyes stains and pigments that may be used individually or as an ingredient in a staining compound include, but are not limited to, are:

carbon black, FD&C Blue #2, nigrosin, FD&C black shade, FD&C blue #1, methylene blue, FD&C blue #2, malachite green, D&C green #8, D&C green #6, D&C green #5, ethyl violet, methyl violet, FD&C green #3, FD&C red #3, FD&C red #40, D&C yellow #8, D&C yellow #10, D&C yellow # 11, FD&C yellow #5, FD&C yellow #6, neutral red, safranine 0, FD&C carmine, rhodamine G, napthol blue black, D&C orange #4, thymol blue, auramine 0, D&C red #22, D&C red #6, xylenol blue, chrysoidine Y, D&C red #4, sudan black B, D&C violet #2, D&C red #33, cresol red, fluorescein, fluorescein isothiocyanate, bromophenol red,

D&C red #28, D&C red #17, amaranth, methyl salicylate, eosin Y, lucifer yellow, thymol, dibutyl phthalate, indocyanine green, and the like. The preferred stain is one that is generally deemed biologically compatible or non-toxic and may include any of the above dyes, pigments and stains as an ingredient in a final solution used as a stain. Other stains, currently existing or discovered or manufactured in the future, may be readily utilized in this method. Therefore, the above listing should not be considered definitive, but rather illustrative of stains to be utilized in the disclosed method and in no way be considered limiting.

One method of applying the stains to biological tissues to be cut or destroyed can be performed by placement of either a powdered or a liquid form directly on the tissues. This can be done by spreading or smearing a dried powder with a flat instrument over the biological tissue to be treated. The soluble stains can be dissolved in a solvent such as water, glycerin, propylene glycol, mineral oil, ethanol, acetone, polysorbate 80, or any like solvent. These dissolved stains can be applied to biological tissues by means of a brush, a syringe, a pen, a cotton pellet, or any fibrous material. Some stains may be a liquid without being dissolved by a solvent; these may also be applied by means of a brush, a cotton pellet, a syringe, a pen, or any fibrous material. Liquid stains may also be injected by means of a hypodermic needle and syringe or any other subcutaneous injection device to a target area beneath the surface of biological tissue. These internal treatment areas can be radiated arthroscopically or with any other subcutaneous method or device with radiant energy. These stains may optionally contain an anesthetic such as lidocaine, benzocaine, or any local or systemic anesthetic that would aid in alleviating any pain or discomfort caused by the procedure. If properly applied, the stain may additionally serve to map a practitioner's intended treatment area and plan, thereby serving a secondary purpose as well as enhancing the utility of the laser treatment. In current practice, the drawn features in a map serve no purpose other than to indicate where a practitioner is to cut and serve as a general guide to the treatment procedure.

These stains can be formulated into various compositions to best fit a medical, veterinary, or dental procedure, examples of which are presented below:

### Example Formula #1

100%—nigrosin

### Example Formula #2

1%—nigrosin

99%—water

### Example Formula #3

100%—FD&C Blue #2

### Example Formula #4

1.5%—FD&C Blue #2

98.5%—water

### Example Formula #5

0.1%—FD&C Blue #2

30%—ethanol

69.9%—Water

### Example Formula #6

1%—FD&C Green #3

30%—ethanol

69%—Water

### Example Formula #7

2%—Cresol red

98%—ethanol

### Example Formula #8

0.5%—amaranth

10%—ethanol

89.5%—glycerol

### Example Formula #9

100% Amaranth

### Example Formula #1 0

1%—Eosin 4-isothiocyanate

25%—Polyethylene glycol 600

74%—ethanol

### Example Formula #11

99%—Bromophenol Red

1%—Water

### Example Formula #1 2

1.0%—FD&C Yellow #5

99%—Glycerol

### Example Formula #1 3

3%—FD&C Blue #2

10%—polysorbate 80

87%—Water

### Example Formula #14

5%—Indocyanine Green

95%—Water

The above example formulas are all able to adequately stain biological tissue. The methods for cutting or destroying tissue warrant use of a radiant energy source with sufficient energy to destroy biological tissue. The radiant energy can be produced from sources such as high intensity light from incandescent, halogen or plasma arc devices. The radiant energy can be produced from sources such as solid-state lasers, examples of which are neodymium YAG, titanium sapphire, thulium YAG, ytterbium YAG, Ruby, holmium YAG lasers and the like. The radiant energy can be produced from sources such as EB or electron beam devices. The radiant energy can be produced from sources such as gas lasers, examples of which are the Carbon dioxide laser, argon gas, xenon gas, nitrogen gas, helium-neon gas, carbon monoxide gas, hydrogen fluoride gas lasers and the like. The radiant energy can be produced from sources such as a diode laser, examples of which are the gallium nitride, aluminum gallium arsenide diode laser and the like. There are also many dye lasers that utilize a radiant energy source that pass through various stains to achieve various wavelengths. Dye lasers are also within the scope of this method. Any wavelength of radiant energy, from 200 nm to 8,000 nm, may be utilized so long as a proper stain is found to match the wavelength emitted by the emitting source.

The method can include use of a radiant energy opaque substance that can be applied around the stained treatment area to protect against incidental or accidental exposure of harmful radiant energy during treatment. A typical procedure would begin by staining the area to be treated with a stain that is attuned to absorb the light from a radiant energy source, followed by covering the surrounding area with a substance that reflects or is opaque to the incoming radiant energy being produced. This combined procedure allows for targeted or selective destruction of biological tissues. The procedure allows the clinician to destroy precisely the biological tissues selected and keep intact those tissues that are intended to remain. A radiant energy opaque substance can be one that reflects most radiant energy and of a substance that is not combustible, for example, inorganic compounds that do not readily combine with atmospheric gases at elevated temperatures. Examples of radiant energy opaque substances are titanium dioxide, zinc oxide, calcium carbonate, and the like. Typically, radiant energy opaque substances are usually visibly white in color.

A method of applying the radiant energy opaque substance to biological tissues can be done by placement of the powdered form directly on the tissues. This can be done by spreading or smearing a dried powder with a flat instrument over the biological tissue to be treated. These substances can be blended in water to form a paste. These opaque suspensions can be applied to biological tissues by means of a brush, a flat instrument, a cotton pellet, a syringe, or any fibrous material. The paste can also contain a suspending aid to avoid settling of solids over time. Examples of suspending aids are sodium carboxy methylcellulose, fumed silica, sodium carboxy ethyl cellulose, precipitated silica, guar gum, and the like.

Radiant energy opaque substances can be formulated into various compositions to best fit a medical, veterinary, or dental procedure, an example of which is presented below:

### Example Formula #1b

50%—powdered titanium dioxide

1%—sodium carboxy methyl cellulose

49%—water

The above example formula would be recognized as adequately able to cover and protect biological tissue from incidental harmful radiant energy.

Another variation of this method is to apply an oxidizing substance to the targeted area before use of the laser. An oxidizing substance is any substance that releases oxygen upon decomposition. The substance decomposes and releases oxygen into the immediately surrounding environment, thereby enhancing the destruction of the targeted tissue. The substance may be applied in addition to the stain or may be a component ingredient of the stain if maintained in a stable form. Oxidizing substances may be organic or inorganic. Potential oxidizing substances that may be utilized in this method include: benzoyl peroxide, T-butyl peroxide, T-butyl peroxide benzoate, potassium nitrate, potassium nitrite, potassium chlorate, potassium chlorite, sodium nitrate, sodium nitrite, sodium chlorate, and sodium chlorite. It should be noted, however, that the use of certain stains, such as indocyanine green, may be so efficient as to render the addition of an oxidizing substance superfluous.

In conceptual testing, a radiant energy source was selected for its ability to adjust output wattage settings nearest those used for soft tissue surgery. The 810 nm Odyssey®NAVIGATOR™ Diode laser from Ivoclar/Vivodent, Inc. was used for this study because of the variable controls and the ease of disposable tips. The laser was set to continuous mode throughout the study. The laser hand piece was mounted onto an adjustable laboratory clamp/stand in order to control the constant tip distance to the soft tissue. A steel pre-measured gauge of 1.5 mm thickness was used to ensure the tip distance was as near a consistency of 1.5 mm from the soft tissue as possible. The soft tissue used in this study was pork loin, which was intended to closely mimic human tissue. The wattage settings used in the test were 0.1, 0.2, 0.3, 0.4, 0.5, 1.0, 2.0, and 3.0. A total of 5 stain groups were selected as test groups: no stain (control group) and FD&C Green #3, FD&C Blue #2, Indocyanine green, and Carbon black. A maximum of 1 minute was selected as the duration of time to determine the carbonization treatment window. The criterion to measure whether the soft tissue achieves a state of carbonization was to examine the time it takes for a gray to black dot to form immediately beneath a weak aiming beam. The study considers the formation of the usual black or gray spots as evidence of carbonization and/or combustion. The formation of a gray to black dot or spot is considered a positive test and the time of initiation is noted. The formation of no spot or dot is a negative test or none formed.

The experiment in general consisted of laying a fairly flat piece of pork loin on a flat surface and positioning the laser tip with the aid of the steel gauge to about 1.5mm from the surface. To the pork loin was then applied a coat of the various stains and subsequently irradiated at the various power settings until carbonization was achieved or 1 minute of time elapsed. The time was controlled with a stopwatch. The following table presents the results:

| | No Stain | FD&C | FD&C | Indocyanine | Carbon |
|---|---|---|---|---|---|
| | (control) | green #3 | Blue #2 | Green (ICG) | Black |
| 0.1 Watt | No | NC | NC | NC | NC |
| | Carbonization | | | | |
| | (“NC”) | | | | |
| 0.2 Watt | NC | NC | NC | NC | NC |
| 0.3 Watt | NC | NC | NC | NC | 58 sec |
| 0.4 Watt | NC | NC | NC | 47 sec | 26 sec |
| 0.5 Watt | NC | NC | NC | 21 sec | 11 sec |
| 1.0 Watt | NC | NC | NC | 5 sec | 1 sec |
| 2.0 Watt | NC | NC | NC | 2 sec | 1 sec |
| 3.0 Watt | NC | NC | NC | 1 sec | 1 sec |

The stains were chosen for their various absorption efficiencies with respect to a λₘₐₓ of 810 nm. The absorption efficiency is merely a percentage of energy absorbed by the stained tissue with respect to energy output. Carbon black was selected as a universal stain with absorption efficiencies above 95% over a wide range of wavelengths; as can be seen from the data how effective it was over the control. Indocyanine Green was selected for its known λₘₐₓ near 810 nm and has absorption efficiency greater than about 90%; it also allowed carbonization of soft tissue at a much lower wattage than an unmatched stain and/or control groups. FD&C Blue #2 was selected for its minimal absorption characteristics at 810 nm, with only about a 30% efficiency it did no better than the control, though it would in theory initiate carbonization sooner than the control at higher wattages. FD&C green #3 was selected because it had insignificant absorption efficiency at 810 nm and as demonstrated—did no better than the control.

The data demonstrates that when the absorption characteristics of a stain are matched to the wavelength of a radiant energy source, the power output required to initiate carbonization is significantly reduced. Carbon black initiated carbonization with as little as 0.3 watts at a distance of 1.5 mm from the pork loin. On the other hand, the control did not initiate carbonization at 3.0 watts at 1.5 mm. This study shows that it is possible to paint any given tissue, regardless of the absorption characteristics of said tissue and carbonize said tissue selectively and at a much lower wattage. It also demonstrates that at these lower wattage settings, unstained tissue will be unharmed by the radiant energy.

An actual in vivo clinical test recently performed confirmed the efficacy of the present invention. In the test, a laser source emitting laser energy having a wavelength of about 810 nm and a power level of about 5 W was used to expose a cancerous tumor having a volume about 9 mm in diameter to laser energy for about 5 minutes. Necrosis of the tumor began after about 1 minute of exposure, and the tumor was substantially destroyed after about 5 minutes, resulting in destruction of all or substantially all of the cancerous cells exposed to the laser energy.

Preferred embodiments will depend upon the laser available to a clinician. However, in each case, the stain should have an absorption efficiency of greater than 90% at the given laser source's λₘₐₓ. Obviously, the higher the efficiency, the lower power output from the laser source will be necessary and less collateral damage to healthy tissue will occur. As illustrated above, for an 810 nm diode laser, carbon black or indocyanine green may be used. In the case of an absorption efficiency of 95% or greater, only .3 W of power may be used as a minimum. At an efficiency of 90% or greater, the power output may be 4 W or greater. Stronger power outputs may be used to lessen treatment time and still not affect untreated tissue as illustrated in the conceptual test. Other dyes may be used so long as they have a λₘₐₓ that allows for an absorption efficiency of 90% or greater for a given wavelength of energy. For example, toluidine blue has a λₘₐₓ at 626 nm, so it may be used with a radiant energy source capable of emitting such energy at that wavelength. Bromophenol blue has three λ-maxima, at 383, 422 and 589 nm respectively, and may be used with a corresponding radiant energy source for either of those three maxima. The actual power output should be left to the clinician to determine based on each particular case, as size and location of the targeted tissue will also factor into treatment times and power output. It is possible for treatment times to extend as little as one minute or as long as an hour or more depending on the wattage used, size of the tissue, absorption efficiency and other factors.

Another embodiment of this invention, shown in FIG. 18, allows the use of radiant energy at wavelengths to which soft tissue of the human body is normally transparent. Various wavelengths are known to which living tissue is transparent, X-rays and gamma ray are such examples. However, radiant energy with a wavelength between 900 and 1100 nm would also pass though living soft tissue normally without causing damage to said tissue. It is possible to stain a tissue mass 501 with stain that absorbs such energies and follow the methods taught herein for destruction of said tissue. The tissue would then be capable of absorbing the radiant energy 502 while residing underneath a column of layers of tissue 503, 504, and 505 transparent to that same energy. In such a treatment, no entry into the living body is necessary as unstained soft tissue would be transparent to the energy as radiant energy will be directed towards the targeted tissue mass 501 and pass through the “transparent” tissue with no harm to that tissue. The tissue mass 501, however, being stained with the appropriate nontransparent stain, will absorb the radiant energy and be destroyed according to the earlier teachings of this invention. Appropriate materials for such “stains” include: amminium dyes as for example metal tris amminium dyes or metal tretrakis amminium dyes wherein the metal includes boron, iron, cobalt, nickel, copper, or zinc such as cobalt tris amminium various metal dithiolene dyes wherein the metal includes boron, iron, cobalt, nickel, copper, or zinc, such as 5 nickel dithiolene, and the like; various diphenylmethane, triphenylmethane and related dyes; various quinone dyes such as naphthoquinone dyes; various azo type dyes; various benzene dithiol type metal complex dyes wherein the metal includes boron, iron, cobalt, nickel, copper, or zinc; various pyrylium type dyes; various squarylium type dyes;

various croconium type dyes; various azulenium type dyes; various dithiol metal complex type dyes; various indophenol type dyes; and various azine type dyes. Exposing hard tissue, such as bone, to such radiant energy should be avoided.

Although the present invention has been described with reference to preferred embodiments, numerous modifications and variations can be made and still the result will come within the scope of the invention. No limitation with respect to the specific embodiments disclosed herein is intended or should be inferred.

I claim:

1. A method for targeted destruction of biological tissues by application of radiant energy, comprising:
a. a step of identifying targeted tissue;
b. a step of selecting a stain with an absorption spectrum corresponding to a wavelength of a given radiant energy source;
c. a step of applying the stain to a biological tissue substrate to be destroyed; and
d. a step of communicating radiant energy to the tumor with sufficient energy to destroy the targeted tissue through at least one process of destruction selected from the group of processes of destruction consisting of carbonization and vaporization, while simultaneously minimizing harm to tissue surrounding the targeted tissue.

2. The method of claim 1, wherein the stain has an absorption efficiency to the radiation energy higher than 80%.

3. The method of claim 1, the radiant energy emitted having a wavelength in the range from about 200 nm to about 8,000 nm.

4. The method of claim 1, wherein the laser system operating at a power level of at least 0.3 Watts.

6. The method of claim 1, wherein the stain is selected from the group consisting of indocyanine green, carbon black, FD&C Blue #2, nigrosin, FD&C black shade, FD&C blue #1, methylene blue, FD&C blue #2, malachite green, D&C green #8, D&C green #6, D&C green #5, ethyl violet, methyl violet, FD&C green #3, FD&C red #3, FD&C red #40, D&C yellow #8, D&C yellow #10, D&C yellow #11, FD&C yellow #5, FD&C yellow #6, neutral red, safranine 0, FD&C 10 carmine, rhodamine G, napthol blue black, D&C orange #4, thymol blue, aurarnine 0, D&C red #22, D&C red #6, xylenol blue, chrysoidine Y, D&C red #4, sudan black B , D&C violet #2, D&C red #33, cresol red, fluorescein, fluorescein isothiocyanate, bromophenol red, D&C red #28, D&C red #17, amaranth, methyl salicylate, eosin Y, lucifer yellow, thymol, and dibutyl phthalate.

7. The method of claim 1, wherein the laser system is selected from the group consisting of semiconductor lasers, solid state lasers, and gas lasers.

8. The method of claim 1, wherein the laser system emits radiant energy of a modulating power level in the range of from 0.1 watt to 30 watts.

9. The method of claim 1, wherein the tissue is exposed to the laser light for a time duration that is within the range of from about 1 second to about 1 hour.

10. The method of claim 1, the stain being applied to the targeted tissue using a syringe.

11. The method of claim 1, the stain being applied to the targeted tissue by spreading a paste containing the stain over the selected biological tissue.

12. The method of claim 1, the stain being applied to the targeted tissue substrate by spreading a powder containing the stain over the selected tissue.

13. The method of claim 1, the stain being applied to the targeted tissue substrate by spreading a liquid containing the stain over the selected tissue.

14. The method of claim 1, the stain being applied to the targeted tissue by utilizing a pen containing the stain to mark the tissue.

15. The method of claim 1, the stain being applied to the targeted tissue intravenously.

16. The method of claim 1, the radiant energy being communicated to the targeted tissue arthroscopically.

17. The method of claim 1, the stain further comprising an anesthetic.

18. The method of claim 1, the stain further comprising an oxidizing substance.

19. The method of claim 18, the oxidizing substance being selected from the group of oxidizing substances consisting of: benzoyl peroxide, T-butyl peroxide, T-butyl peroxide benzoate, potassium nitrate, potassium nitrite, potassium chlorate, potassium chlorite, sodium nitrate, sodium nitrite, sodium chlorate, and sodium chlorite.

20. The method of claim 1, further comprising a step of applying a radiant opaque substance to tissues surrounding the biological tissue substrate, wherein said surrounding tissues are then protected from absorbing energy from the radiant source.

21. The method of claim 1, the laser system emitting radiant energy at a wavelength to which living tissue is transparent and the radiant energy passes through an uninterrrupted column of unstained tissue to reach the targeted tissue.

22. The method of claim 21, the wavelength of the radiant energy being within the range between 900 and 1100 nm, inclusively.

23. The method of claim 22, the stain being selected from the group of stains consisting of amminium dyes, metal tris amminium dyes, metal tretrakis amminium dyes, metal dithiolene dyes, benzene dithiol type metal complex dyes, wherein the metal includes boron, iron, cobalt, nickel, copper, or zinc; diphenylmethane; triphenylmethane; quinone dyes; azo type dyes; pyrylium type dyes; squarylium type dyes; croconium type dyes; azulenium type dyes; dithiol metal complex type dyes; indophenol type dyes; and azine type dyes.
